(19) United States
(12) Patent Application Publication
(10) Pub. No.: US 2025/0143886 A1
(43) Pub. Date: May 8, 2025

(54) Cemented Sheath For Joint Implant

(71) Applicant: Howmedica Osteonics Corp., Mahwah, NJ (US)

(72) Inventors: Ogden Myklebust, Friendswood, TX (US); Alexander Slayton, Denville, NJ (US); Josh Peterson, Warwick, NY (US); Peter Tulkis, Paramus, NJ (US)

(21) Appl. No.: 18/938,976

(22) Filed: Nov. 6, 2024

Related U.S. Application Data
(60) Provisional application No. 63/547,776, filed on Nov. 8, 2023.

(51) Int. Cl. A61F 2/34

(52) U.S. Cl. CPC A61F 2/34; A61F 2002/3443; A61F 2002/348

(57) ABSTRACT

A prosthetic implant assembly including a modified liner configured for fixation to an acetabular shell. The modified liner having a sheath and a liner. The liner is configured to be inserted into and affixed to the sheath. The sheath having an annular rim, a plurality of longitudinal ribs extending from the annular rim, and a cross-rib spaced apart from the annular rim and intersecting the plurality of longitudinal ribs. Each longitudinal rib of the plurality of longitudinal ribs is curved such that the plurality of longitudinal ribs collectively define a partially spherical surface. Additionally, the annular rim, the plurality of longitudinal ribs, and the cross-rib define a plurality of openings between the annular rim and the cross rib.

### CROSS-REFERENCE TO RELATED APPLICATIONS

This application claims the benefit of U.S. Provisional Patent Application No. 63/547,776, filed Nov. 8, 2023, the disclosure of which is hereby incorporated by reference.

### BACKGROUND

Ball and socket prosthetic implant assemblies are designed to allow for biomechanical movement while remaining securely assembled and tightly fitted within a joint of a patient. Providing an implant with such characteristics can be challenging with patients that are young and mobile and with patients that have bone loss and poor bone quality around the joint, particularly where a revision surgery is needed. In such cases, one priority for surgeons is to ensure that the shell for the socket of the joint is strongly fixed to the bone, even if it is necessary to compromise in terms of having a sub-optimal orientation of the shell to obtain such fixation. While in most cases, the liner orientation follows the orientation of the shell to ensure a strong connection between the shell and the liner, in cases where the liner is oriented in a sub-optimal fashion the resultant biomechanics of the patient may be disrupted and otherwise irregular, increasing the risk of dislocation post-surgery, and also leading to a higher likelihood of a need for revision surgery.

Thus, further improvements are desired in the field of ball and socket joint implant assemblies so that such assemblies provide strong anchorage to bone and a desired range of motion. Additionally, another improvement desired is the provision of implant assembly designs that reduce the number of implant components needed to carry out a specific procedure.

### BRIEF SUMMARY

In a first aspect, the present disclosure relates to a prosthetic assembly that includes a sleeve that is disposed on a liner and within a cup-like shell such that bone cement may be used to anchor the sleeve and liner combination onto the shell. The sleeve may be of a modular design and formed monolithically. In a first embodiment, the sleeve has a generally hemispherical shape and includes a network of interconnected segments extending longitudinally from an annular ring toward a polar region of the sleeve. In some examples, the annular ring may include a plurality of spaced apart notches and/or protrusions for engagement to the liner. In other examples, the annular ring may include a threaded opening at the pole. In still further examples, the sleeve may include clips extending from the annular ring. In some examples, the sleeve may define a hemispherical structure similar to a colander-like bowl. In operation, the annular ring of the sleeve is fitted around a liner such that the sleeve securely attaches to the liner by engaging respective notches and or protrusions, threading onto a threaded post extending from the liner, or hooking onto the liner with clips that engage the rim of the liner. In a second embodiment, a sleeve may have a truncated hemispherical structure with first and second annular apertures disposed opposite of each other. In the above embodiments, the sleeve is modular in that it can be used to secure any one of a variety of liners having different shapes and sizes to a shell. For example, the sleeve can modify liners that may otherwise be too small to securely fit within a given acetabular shell. In some examples, the sleeve is paired with a modular dual mobile liner.

In a second aspect, the present disclosure relates to a prosthetic implant assembly having a modified liner. The modified liner may be configured for use in a hip joint and may include a sheath and a liner. The sheath may have an annular rim, a plurality longitudinal ribs extending from the annular rim, and a first cross-rib spaced apart from the annular rim and intersecting the plurality of longitudinal ribs such that the first cross-rib defines a closed loop. Each of the longitudinal ribs of the plurality of longitudinal ribs may be curved such that the plurality of longitudinal ribs collectively define a partially spherical surface. The annular rim, the plurality of longitudinal ribs, and the first cross-rib may define a plurality of openings between the annular rim and the first cross rib. The liner may be configured to be received in the sheath. In such embodiments, the sheath may include second cross-rib defining a closed loop and spaced apart from the first cross-rib such that the second cross-rib intersects the plurality of longitudinal ribs. In yet other embodiments, the plurality of longitudinal ribs, the first cross-rib and the second cross-rib may define a webbed structure such that a majority of the plurality of openings are quadrilateral.

In some other embodiments, the sheath may further comprise a plurality of cross-ribs in addition to the first cross-rib such that each cross-rib of the first cross-rib and the plurality of cross-ribs being evenly spaced with respect to an adjacent cross-rib. In other embodiments, each end of each longitudinal rib of the plurality of longitudinal ribs may be attached to the annular rim of the sheath, and the plurality of cross-ribs extend circumferentially around the sheath. In yet other embodiments, the cross-ribs may vary in distance from the annular rim along its length. In yet some other embodiments, each opening of the plurality of openings may be a cuboid shape. In other embodiments, each opening of the plurality of openings may have a first outer perimeter shape. In yet another embodiment, the annular rim may have a band extending therefrom. In still yet another embodiment, in a direction of a length of the longitudinal rib, a dimension of the band may be greater than each of the cross-ribs. In some embodiments, the sheath may have a hemispherical shape. In yet other embodiments, the annular rim may be located at an equatorial region of the hemispherical shape.

In other embodiments, the sheath may be a truncated hemisphere having a first end opening and a second end opening such that the first end opening defines an inner diameter of the annular rim with the inner diameter of the annular rim being larger than that of the second opening. The second end opening may define a jagged edge configured to flex around the liner inserted therein. In some embodiments, the first and second end openings are concentric. The liner may include a lip that extends over the annular rim of the sheath.

In accordance with another aspect, the present disclosure relates to a process for implanting a prosthetic implant. This process may include an acetabular shell that is inserted into an acetabular cavity of a pelvis. After such insertion, an adhesive may be applied on one of the acetabular shell or a modified liner. Once adhesive is applied, the modified liner may be inserted into the acetabular shell. In such instances, prior to insertion of the liner, the liner may be oriented such that the annular rim of the sheath is angled with respect to a plane defined by a rim of the acetabular shell. In this and other instances, the adhesive may be a bone cement that envelopes a shell-facing surface of the sheath of the modified liner. Additionally, the liner may be attached to the sheath before inserting the modified liner into the acetabular shell.

In accordance with another aspect, the present disclosure relates to a process of reconstructing an acetabular implant assembly. This process may include a preexisting liner disposed within an acetabular shell within a patient that is removed. Once this is done, an adhesive may be placed on one or both of the acetabular shell and a modified liner comprising a replacement liner and a sheath disposed over the replacement liner. Once adhesive is placed, the modified liner may be positioned onto a cavity surface within the acetabular shell such that the modified liner and acetabular shell are joined through the adhesive. The adhesive passes through the sheath via each opening of a plurality of openings through the sheath when the plurality of openings are located on a portion of the sheath between an equatorial rim of the sheath and a polar region of the sheath. In this and other instances, orienting the modified liner may include the modified liner being positioned such that a first plane through at least three separate locations on the annular rim of the sheath is angled with respect to a second plane through at least three separate locations on a rim of the acetabular shell. In another instance, orienting the modified liner may include the modified liner being positioned such that a first plane through at least three separate locations on the annular rim of the sheath is parallel to a second plan through at least three separate locations on a rim of the acetabular shell. Additionally, the placement of the adhesive may include bone cement being placed on one or both of the acetabular shell and a modified liner. In other instances, positioning of the modified liner may include the modified liner being positioned at an angle relative to the acetabular shell that is different from a preoperative angle of the preexisting liner relative to the acetabular shell. The replacement liner selected may be a different size than that of the preexisting liner. Removing the preexisting liner may include a cementless connection between the acetabular shell and the preexisting liner being disassembled.

In accordance with yet another aspect, the present disclosure relates to a prosthetic implant assembly that includes a sheath and a liner. The sheath may have an annular rim with a first diameter, a plurality of longitudinal ribs each extending along a curve from the annular rim, and a cross-rib with a second diameter intersecting the plurality of longitudinal ribs. The second diameter may be smaller than the first diameter. An implant-receiving side of each longitudinal rib of the plurality of longitudinal ribs may define a portion of a cavity such that each longitudinal rib of the plurality of longitudinal ribs may have a convex side opposite the implant-receiving side. The plurality of longitudinal ribs and cross-rib may define a plurality of intersection points between the plurality of longitudinal ribs and the cross-rib. The liner may be configured to be received into the cavity of the sheath. Additionally, an implanted acetabular shell may be configured to receive a modified liner comprising the sheath and the liner. In this and other instances, the sheath may include a plurality of cross-ribs. The cross-rib may include a plurality of U-shaped segments.

In yet other instances, the annular rim, the plurality of longitudinal ribs, and the cross-rib define a plurality of openings between the annular rim and the cross-rib. Each opening of the plurality of openings may have the same dimensions. The liner may be a modular dual mobility liner. The liner may include a side protrusion disposed on a shell-facing surface and near a rim of the liner. The liner and the sheath may be connected via the side protrusion. The sheath may include a plurality of lips extending from the annular rim of the liner such that the plurality of lips can be configured to grip onto the liner to hold the sheath relative to the liner. The liner may be threadably attached to the sheath. The liner may include a plurality of liner-removal tabs that extend radially outward. The annular rim of the sheath may include a plurality of notches each configured to receive one liner-removal tab of the plurality of liner-removal tabs. The sheath may be a hemispherical shape. The annular rim may be located at an equatorial region of the hemispherical shape. The sheath may be a truncated hemisphere having a first aperture and a second aperture such that the first aperture defines an inner diameter of the annular rim, and the inner diameter of the annular rim may be larger than that of the second aperture. The second aperture may define a jagged edge configured to flex. The first aperture and the second aperture may be concentric. The liner may include a ledge that extends over the annular rim of the sheath. When the liner is received and secured in the sheath and the sheath is secured in the acetabular shell, the liner may be fixed in place at an angle with respect to the acetabular shell.

In accordance with yet another aspect, the present disclosure relates to a process of implanting a prosthetic implant. This process may include a liner and a sheath being retrieved. The liner may be inserted into the sheath to form a modified liner. Once the liner is inserted, an adhesive may placed on one of an acetabular shell and/or the modified liner. Then the modified liner may be inserted into the acetabular shell. Additionally, the modified liner may be oriented such that the annular rim of the sheath is angled with respect to a plane defined by a rim of the acetabular shell. The adhesive may be a cement that covers at least part of a shell-facing surface of the sheath of the modified liner. The liner may be placed into the sheath before inserting the modified liner into the acetabular shell.

In accordance with another aspect, the present disclosure relates to a kit for acetabular implant revision. This is kit may include a plurality of liners and a sheath. At least two of the liners of the plurality of liners may be a different size from each other. The sheath may be adapted to receive each liner of the plurality of liners such that the sheath may have an at least partial cup shape with a cavity-facing surface and a convex surface opposite the cavity-facing surface and a convex surface opposite the cavity-facing surface. The sheath may also have a plurality of first openings extending circumferentially parallel to an annular rim of the sheath and a plurality of second openings extending circumferentially parallel to the annular rim. The plurality of first openings may be at a different distance from the annular rim compared to the plurality of second openings and the plurality of first openings may be different from the plurality of second openings. Each of the plurality of liners may be a different size and/or shape. Each opening of the plurality of first and second openings may be circular in shape. Each hole of the plurality of holes may be coaxially aligned with another hole of the plurality of holes. Additionally, surgical tools may be included that are configured to remove at least one liner of the plurality of liners from an existing acetabular shell implant. An adhesive may be included that is configured to secure the sheath and a liner to an implanted acetabular shell. A plurality of sheaths may be included in the kit as well.

In accordance with another aspect, the present disclosure relates to a sheath for use in a ball-and-socket joint of a mammalian body. The sheath may include an annular band, a plurality of longitudinal ribs, and a first annular cross-rib. The annular band may have an edge defining a rim of the sheath. The plurality of longitudinal ribs may each be extending in an arcuate manner from the annular band. The first annular cross-rib may be connected to each longitudinal rim of the plurality of ribs such that the first annular cross-rib may be spaced apart from the annular band. The first annular cross-rib may be narrower than the annular band. The sheath may also include a plurality of through openings with each opening of the plurality of through openings being defined by the annular band, two longitudinal ribs of the plurality of ribs, and the first annular cross-rib. The sheath may be adapted to be received on a convex surface of a ball-shaped implant for the ball-and-socket joint.

The present disclosure also relates to a prosthetic implant assembly that may include an acetabular shell, a sheath, and a liner configured to receive the sheath and the liner attached together. The annular rim of the sheath may have a modular flange extending away from the sheath. The modular flange may include a plurality of holes for bone screws. The annular band may define a groove on a liner-facing surface of the band. The sheath may be made from a polymeric material. The sheath may be made from a metallic material. The sheath may include a second annular cross-rib defining a closed loop and spaced apart from the first annular cross-rib such that the second annular cross-rib may intersect the plurality of longitudinal ribs. The first annular cross-rib may vary in distance from the annular band along its length. Each opening of the plurality of through openings may be a cuboid shape. Each opening of the plurality of through openings may have a first outer perimeter shape.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a perspective view of a sheath in accordance with one embodiment of the present disclosure;

FIG. 2 is a perspective view of the sheath of FIG. 1 disposed on a liner;

FIG. 3 is a cross sectional view of a prosthetic implant assembly according to one embodiment of the present disclosure that includes the sheath of FIG. 1;

FIG. 4 is a cross sectional view of a prosthetic implant assembly according to one embodiment of the present disclosure that includes the sheath of FIG. 1;

FIG. 5 is a perspective view of a sheath in accordance with one embodiment of the present disclosure;

FIG. 6 is a perspective view of the sheath of FIG. 5 disposed on a liner;

FIG. 7 is a perspective view of a sheath in accordance with one embodiment of the present disclosure;

FIG. 8 is a perspective view of the sheath of FIG. 7 disposed on a liner;

FIG. 9 is a cross sectional view of a prosthetic implant assembly according to one embodiment of the present disclosure that includes the sheath of FIG. 7;

FIG. 10 is a cross sectional view of a prosthetic implant assembly according to one embodiment of the present disclosure that includes sheath of FIG. 7;

FIG. 11 is a perspective view of a sheath in accordance with one embodiment of the present disclosure;

FIG. 12 is a perspective view of the sheath of FIG. 11 disposed on a liner;

FIG. 13 is a cross sectional view of a prosthetic implant assembly that includes the sheath of FIG. 11 according to one embodiment of the disclosure;

FIG. 14 is a perspective view of a sheath in accordance with one embodiment of the present disclosure;

FIG. 15 is a top view of the sheath of FIG. 14;

FIG. 16 is a cross sectional view of FIG. 15 across line A-A;

FIG. 17 is a perspective view of the sheath of FIG. 14 disposed around a liner;

FIG. 18 is a perspective view of a sheath in accordance with one embodiment of the present disclosure;

FIG. 19 is a cross sectional view of a prosthetic implant assembly in accordance with one embodiment of the present disclosure;

FIG. 20 is a perspective view of a prosthetic implant assembly in accordance with one embodiment of the present disclosure;

FIG. 21 is a flow chart that shows the steps of implanting a prosthetic implant assembly according to one embodiment of the disclosure; and

FIG. 22 is a flow chart that shows the steps of a revision surgery according to one embodiment of the disclosure.

### DETAILED DESCRIPTION

As used herein unless stated otherwise, the term “proximal” means closer to the heart and the term “distal” means further from the heart. The term “anterior” means toward the front part of the body, and the term “posterior” means toward the back part of the body. The term “medial” means closer to or toward the midline of the body, and the term “lateral” means further from or away from the midline of the body. The term “inferior” means closer to or toward the feet, and the term “superior” means closer to or toward the crown of the head. In addition, the terms “about,” “generally,” and “substantially” are intended to mean that slight deviations from absolute are included within the scope of the term so modified.

While the present disclosure will now be described in detail with reference to the figures, it is done so in connection with the illustrative embodiments but is not limited by the particular embodiments illustrated in the figures.

In one aspect, the present disclosure relates to an apparatus in the form of a sheath, examples of which are shown in the figures, or a larger assembly that includes the sheath. The sheath may be of a modular design and may be formed monolithically. In some examples, the sheath may be designed to be included as part of a ball and socket prosthetic implant assembly. The ball and socket assemblies contemplated for use with the sheath include those adapted for the shoulder and those adapted for the hip. When used in a hip, such prosthetic implant assembly may include one or more of an acetabular shell, a liner, and a femoral head prosthesis. The sheath may be used in both primary surgeries and revision surgeries, as described in greater detail in the methods of the present disclosure.

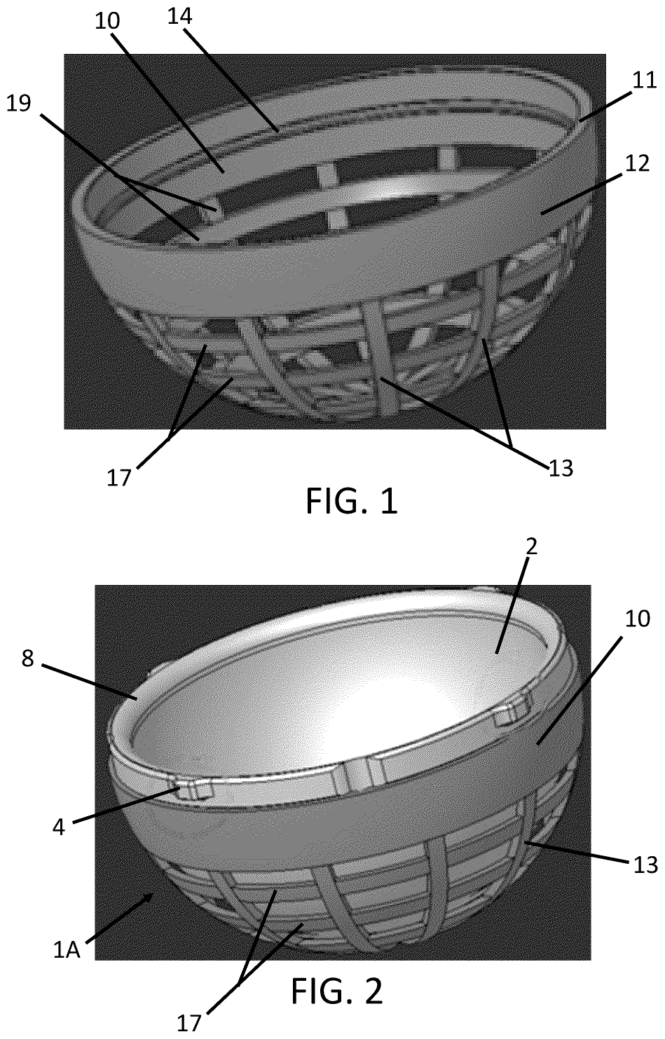

In one embodiment, as shown in FIG. 1, a sheath 10 includes an annular rim 11, a plurality of longitudinal ribs 13 extending from the annular rim, and a plurality of cross-ribs 17 each defining a closed loop, the longitudinal ribs and cross-ribs defining a partially spherical surface and a plurality of openings as the they intersect each other. Further, longitudinal ribs and cross-ribs are evenly spaced apart and have a convex side that is opposite to a liner-facing side 19 (i.e., the implant-receiving side), the liner-facing side defining a hemispherical cavity. Put another way, each longitudinal rib of the plurality of longitudinal ribs 13 extends from the annular rim to a pole such that the plurality of longitudinal ribs define an enclosure. The longitudinal ribs and cross ribs may also be unevenly spaced apart. Each rib of the longitudinal ribs and cross-ribs is curved along a single plane so that the openings defined by the intersecting ribs is cuboid in shape with outer perimeters of each opening being quadrilateral. Annular rim 11 has a band portion 12 on one side of the annular rim extending circumferentially with the annular rim. In this manner, band portion 12, as with annular rim 11, is ring shaped. A width of band portion 12 is greater than a width of longitudinal ribs 13 and cross-ribs 17. Additionally, a cavity-facing side of band portion 12 has a groove 14 that extends around the circumference of the band portion on the liner-facing side of sheath. In some applications, groove 14 of sheath 10 may be complementary to a projection (e.g., a circumferential bead) on a liner so that sheath 10 may interlock with the liner.

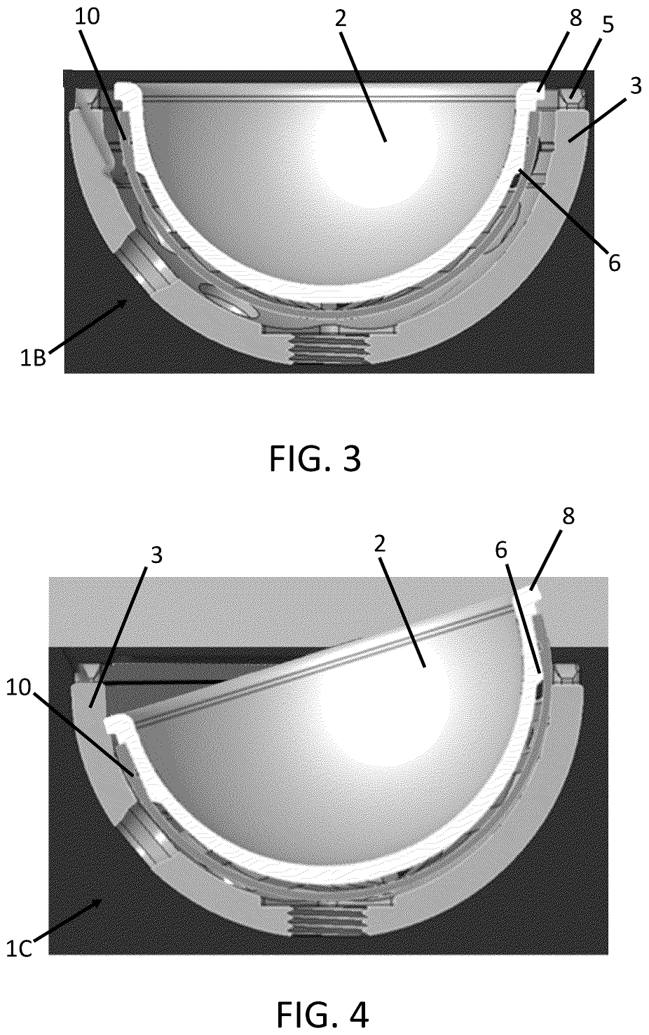

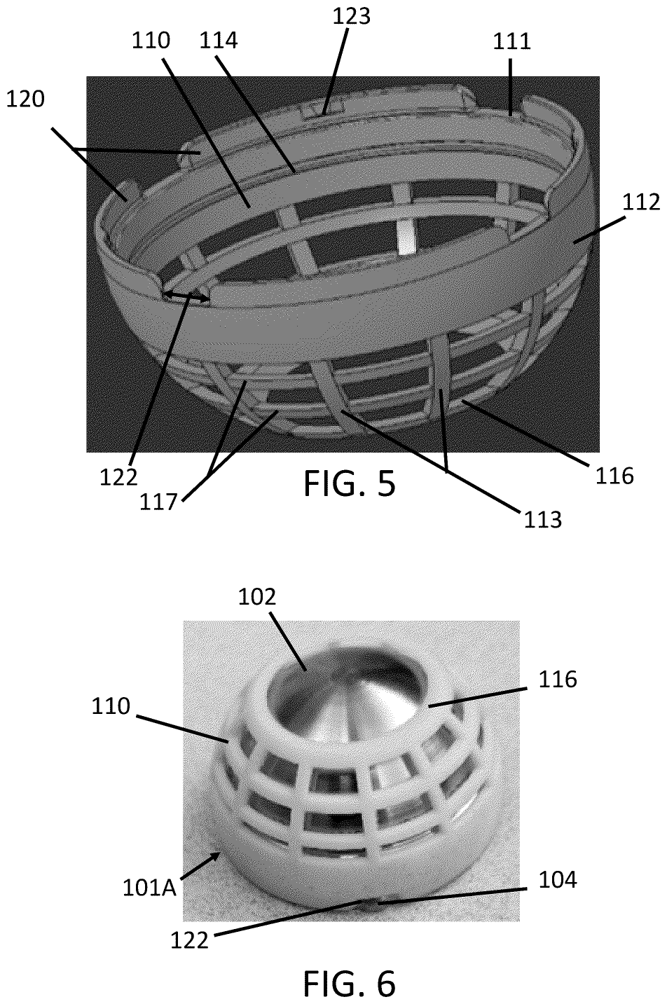

In another embodiment, as shown in FIG. 2, a modified liner assembly 1A includes sheath 10 and liner 2. Liner 2 is disposed within and attached to sheath 10 such that annular rim 11 of the sheath is positioned beneath top rim 8 of liner 2, the top rim having a ledge the extends radially outward over the annular rim of the sheath. Liner 2 has a side protrusion 6, shown in FIGS. 3-4, on an outer surface of the liner adjacent to top rim 8 of the liner that also extends circumferentially around the liner. Protruding from such side protrusion 6 at intervals around a perimeter of the liner are liner-removal components 4 that extend radially outward from an outer surface of the liner. When liner 2 is received in sheath 10, the inward or cavity facing side of band portion 12 of sheath 10 is configured to engage with side protrusion 6 of liner 2. In a variation, the modified liner assembly 1A may have an annular rim such as that included in modified liner assembly 101A shown in FIG. 5.

In other embodiments, a modified liner assembly may be combined with a shell to form a larger implant. Examples of this are shown in FIGS. 3-4 in the form of prosthetic implant assemblies 1B and 1C. Implant assemblies 1B and 1C both include a shell 3, a liner 2, and a sheath 10 attached to the liner so that upon assembly, the sheath is disposed between the shell and the liner. Shell 3 has a generally solid hemispherical or bowl-like shape that includes a shell rim 5. Optionally, and as depicted in FIGS. 3-4, shell 3 includes a plurality of through holes which may or may not be threaded. Such holes may receive fasteners or may serve other functions. The sheath and the liner may be the same as those described with respect to FIGS. 1 and 2. The inclusion of sheath 10 as part of the implant assembly allows liner 2 to be set and oriented at a fixed angle relative to shell 3. Such fixation may be accomplished through the deposit of bone cement or another adhesive in between the modified liner and the shell so that the adhesive creates a bond between the liner and the shell through the openings in the sheath, e.g., openings between longitudinal and cross-ribs. Such setting of the liner at a fixed angle relative to the shell allows an implant to have a customized angulation that is optimized for the biomechanics of a patient. For example, as shown in FIG. 3, the liner of assembly 1B is aligned with the shell. In contrast, in FIG. 4, the liner of assembly 1C is set in place at an angle relative to the shell. This angle may be any angle desired by a user. For example, any angle in a range from 0 degrees up to 90 degrees. While sheath 10 is shown to be compatible with a specific shell and liner in the figures, it should be understood that the sheath has a modular design that will allow it to be used with a large variety of shells and liners of different shapes and sizes.

In another embodiment, sheath 110 is shown in FIG. 5. Sheath 110 is similar to sheath 10 in that sheath 110 has an annular rim 111, a annular band portion 112, a plurality of longitudinal ribs 113, and a plurality of cross-ribs 117. Reference numerals of the 100 series of numerals refer to like elements in the 1 and 10 series of numerals, unless otherwise noted. Sheath 110 has a truncated hemispherical structure. Specifically, sheath 110 includes a first aperture and a second aperture, the first aperture being defined by an annular rim 111 and the second aperture being defined by a superior cross-rib 116. The first aperture at the annular rim has a larger diameter than the second aperture, and the planes through the first aperture and the second aperture are parallel with each other. In this arrangement, longitudinal ribs 113 have a length that extends from band portion 112 to superior cross-rib 116, as shown in FIG. 5 and thus is entirely between the first and second apertures. Further, as with sheath 10, each rib of the longitudinal ribs and cross-ribs is curved along a single plane so that the openings defined by the intersecting ribs is cuboid in shape with outer perimeters of each opening being quadrilateral. Compared to sheath 10, sheath 110 requires less material for fabrication due to its truncated shape without structure in a polar region.

In yet another embodiment, sheath 110 is combined with a liner to form a modified liner 101A, as shown in FIG. 6. Modified liner 101A includes sheath 110 and liner 102. The structural design of liner 102 is the same as liner 2 described above. Liner 102 is disposed through both the first and second apertures of sheath 110, as shown in FIG. 6. Additionally, band 112 of sheath 110 includes a plurality of tabs 120 extending towards the opening and away from the remainder of band 112 and a plurality of recesses 122 disposed between each of the tabs thereby separating the tabs. The recesses are arranged such that each recess can receive a liner-removal component, e.g., projection 104 of liner 102, extending radially outward from liner 102, as shown in FIGS. 5-6. Additionally, a cavity-facing surface of band 112 also includes a plurality of spaced apart notches 123 thereon at the annular rim 111 of the sheath. Each notch of the plurality of notches 123 may engage with a respective projection 104 on an outer surface of the liner annular rim 111. As described in greater detail in methods of assembly described elsewhere in the present disclosure, the liner may be impacted over the sheath in such position to interdigitate the components. In this manner, the plurality of recesses and notches facilitate fixation of the sheath 110 and liner 102 such that they are securely attached to each other. When assembled as modified liner 101A, liner is ready for use with an acetabular shell and may be positioned relative to the shell in a manner desired by the user, as shown, for example, in FIGS. 3 and 4.

In another embodiment, a prosthetic implant assembly includes a shell (not shown) and modified liner 101A inclusive of sheath 110 assembled with liner 102. Shell may be a bowl-like structure similar to shell 3 described above. Like sheath 10 shown in FIGS. 3-4, sheath 110 allows liner 2 to be oriented independent of a shell in a manner that is optimized for the biomechanics of a patient receiving the implant. To be clear, modified liner 101A may be included as part of a prosthetic implant assembly in the same ways as described for modified liner 1A.

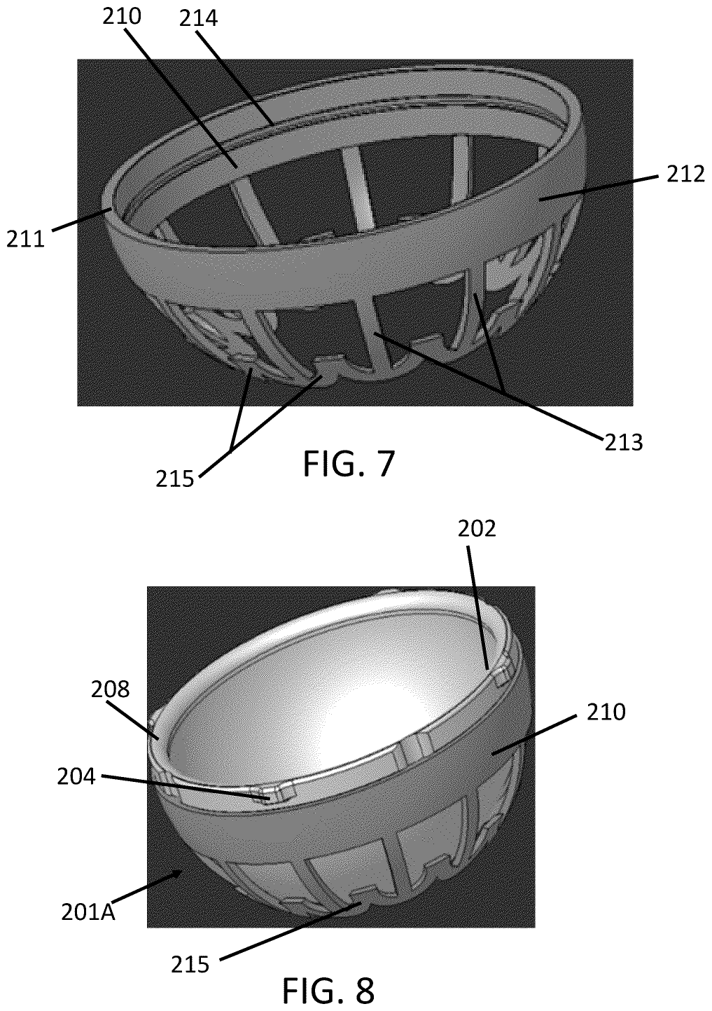

In yet another embodiment, sheath 210 is shown in FIG. 7. Reference numerals in the 200 series of numerals refer to like elements in the 1 and 10 series of numerals, unless otherwise noted. Sheath 210 includes an annular rim 211, a band portion 212, a plurality of longitudinal ribs 213 and a single cross-rib 215. Sheath 210 has a truncated hemispherical structure, similar to sheath 110. As with sheath 110, such truncated hemispherical shape of sheath 210 is bounded by a first aperture defined by an annular rim 211, and a second aperture defined by cross-rib 215. The second aperture has a smaller diameter than that of the first aperture as a curvature of the plurality of longitudinal ribs 213 brings the ribs closer together moving toward a pole from an equatorial region at band portion 212. In sheath 210, cross-rib 215 is, overall, generally parallel to band portion 212 as shown in FIG. 7, but has a repeating wavy pattern along is circumferential dimension. Specifically, cross-rib 215 includes a plurality of adjacent U-shaped segments along its length around a closed loop perimeter. A base of each U-shape intersects an end of a respective longitudinal rib 213. In this arrangement, the shape of the cross-rib 215 inward into the second aperture provides the second aperture with a star-like shape. Longitudinal ribs 213 are spaced apart at equal intervals and extend between band portion 212 and the base of a respective U-shaped segment of cross-rib 215, as noted above. The U-shaped segments have a concave cavity-facing surface (i.e., liner-facing surface) that is shaped to fit and flex around a liner inserted into sheath 210.

In another embodiment, shown in FIG. 8, a modified liner assembly 201A includes sheath 210 and liner 202. Liner 202 has a similar structural design as liner 2 described above. Sheath 210 is designed to be received on and affix to liner 202 in a similar manner as the other sheaths and liners described herein such that sheath 210 connects and engages with exterior features of liner 202.

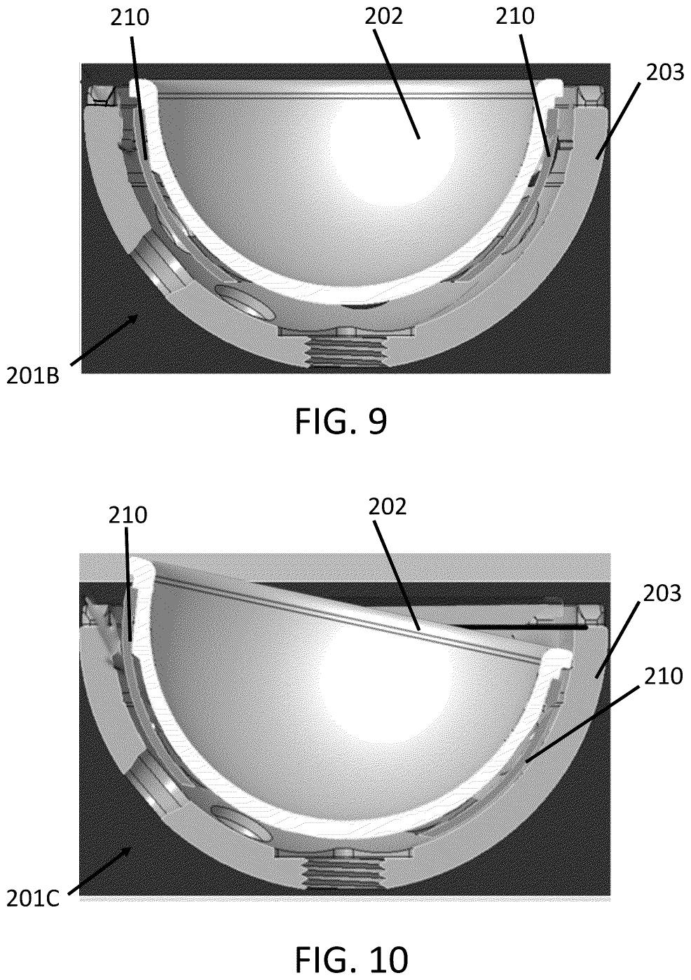

In other embodiments, modified liner assembly 201A may be combined with a shell to form a larger implant. Examples of this are shown in FIGS. 9-10, in the form of prosthetic implant assemblies 201B and 201C. Prosthetic implant assemblies 201B, 201C include a shell 203, a liner 202, and a sheath 210 attached to the liner and disposed between the shell and the liner. Sheath 210 allows liner 202 to be oriented independent of shell 203. For example, in implant assembly 201C shown in FIG. 10, sheath 210 and liner 202 are set in place at an angle with respect to the orientation of shell 203. Alternatively, in implant assembly 201B shown in FIG. 9, modified liner 201A may have the same orientation as shell 203. While these figures represent just two orientations of the modified liner relative to the shell, it should be appreciated that that the modified liners described herein can be tilted and slanted at an angle within the shell as necessary. For example, any angle in a range from 0 degrees up to 90 degrees which is desired by the user. A suitable angle that should meet requirements for post-operative function may be dependent on the natural kinematics of the patient, i.e., how high can the angle be before the risk of impingement is no longer acceptable. Additionally, expected load transfer capacity, i.e., ability to withstand normal wear over a long time period without failure, based on the extent of engagement between the implant components is also a factor. For instance, as the angle between the modified liner and the shell increases, there is less surface area for cementing the two components together, which may, above a certain angle, reduce the load transfer capacity of the assembly. Through the custom angulation between the modified liner and the shell, the modified liners may be better suited for the biomechanics of a patient. Additionally, while sheath 10 is shown to be compatible with a specific shell and liner in the figures, it should be understood that the sheath may have a modular design that will allow it to be used with a large variety of shells and liners of different shapes and sizes.

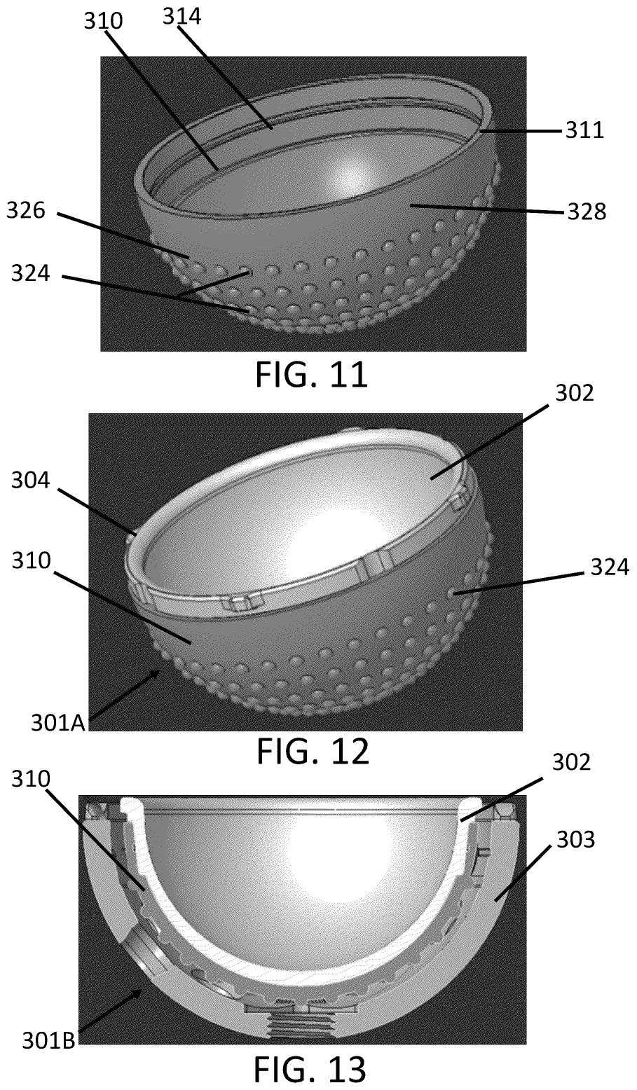

In another embodiment of the sheath, sheath 310 is shown in FIG. 11. Reference numerals in the 300 series of numerals refer to like elements in the 1 and 10 series of numerals, unless otherwise noted. Sheath 310 has a bowl-like structure and includes a plurality of spaced apart protrusions 324 on a convex shell-facing surface 326 of sheath 310. As depicted, sheath 310 has a solid body throughout its bowl-like, e.g., hemispherical shape without any openings in the outer surface. Each protrusion of the plurality of protrusions 324 is a round nub that extends radially outward from outer surface 326. The plurality of protrusions 324 are aligned in parallel circumferential rows that wrap around the outer surface. The same protrusions also define parallel longitudinal columns around the sheath. While protrusions 324 in FIG. 11 are shown as being evenly spaced apart, it is contemplated that such protrusions may be arranged in other patterns or may be distributed randomly in a non-uniform pattern. Sheath 310 also includes a smooth surface region 328 that extends circumferentially adjacent to an annular rim 311. Such smooth surface region 328 is absent any protrusions. The protrusions on sheath 310 are advantageous in that they resist rotation between liner 302 and shell 303 and thus prevent the liner from rotating out of the shell. It should be understood that variations of the other embodiments of the sheath described herein may also include protrusions 324 as included in sheath 310.

In yet another embodiment, shown in FIG. 12, a modified liner assembly 301A includes liner 302 and sheath 310 attached thereon. Liner 302 has a similar structural design as liner 2, 102, 202 described above. Sheath 310 is designed to be receive and affix to liner 302 in a similar manner as the other sheaths 10, 110, 210 and liners 2, 102, 202 described herein such that sheath 310 connects with exterior features of liner 302. In this manner, sheath 310 is combined with liner 302 to create a modified liner configured to be implanted into a shell similar to the other modified liners described herein.

In another embodiment, shown in FIG. 13, a prosthetic implant assembly 301B includes shell 303 and a modified liner 301A that includes liner 302 and sheath 310 disposed thereon. As with other implant assembly embodiments, modified liner 301A is fixed in place relative to shell 303. Securement is enhanced by the plurality of protrusions 324 on sheath 310 that facilitate engagement with an inner surface of shell 303 and any adhesive placed therebetween and thereby resist movement when modified liner 301A is inserted into shell 303. Similar to modified liner 1C shown in FIG. 4, sheath 310 and liner 302 may be set at a fixed angle with respect to the orientation of shell 303. In the example shown in FIG. 13, fixation of the components is such that the modified liner and shell are in alignment and have the same orientation. In other examples, the modified liner may be fixed at an angle relative to the shell, as described for prosthetic implant assembly 1C, for example. In this manner, the orientation of modified liner 301B within shell 303 may be optimized for the biomechanics of a patient.

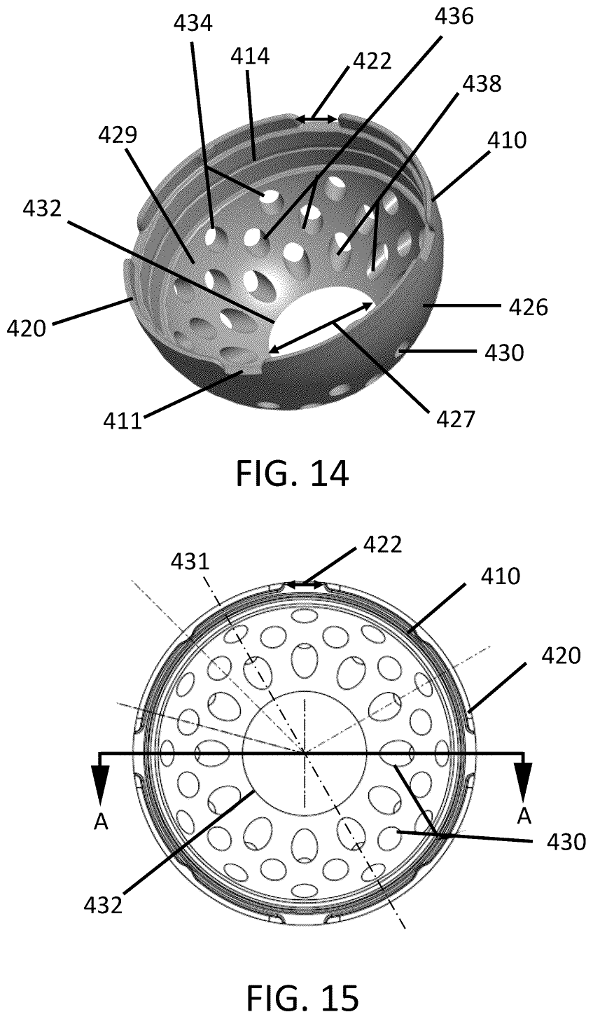

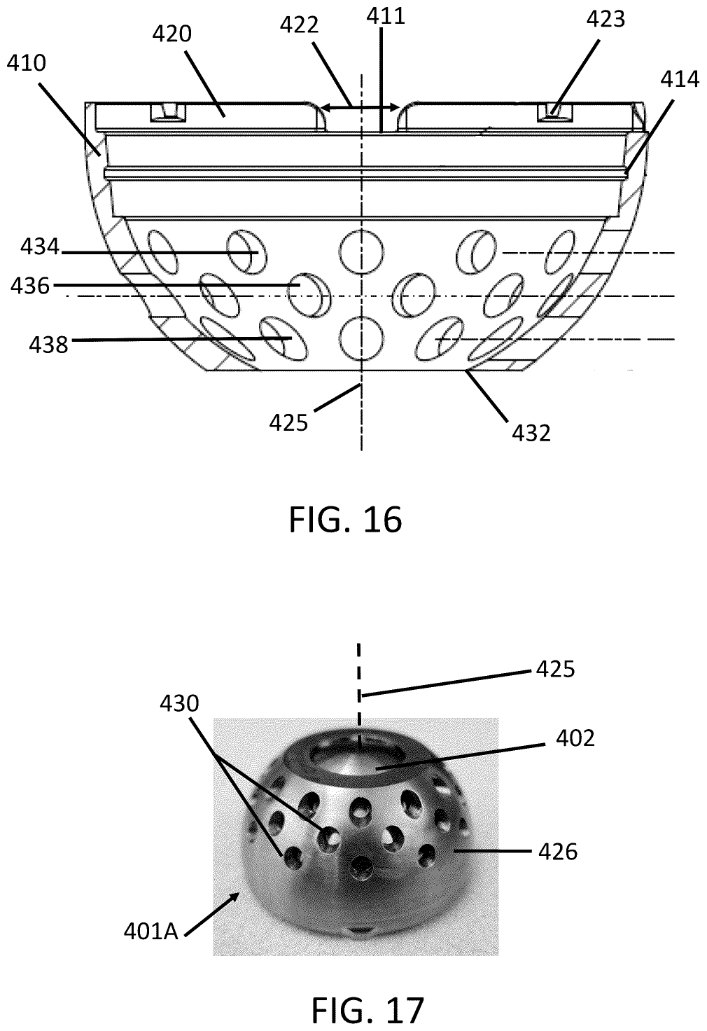

In yet another embodiment, sheath 410, shown in FIGS. 14-16, has a generally hemispherical shape. Reference numerals in the 400 series of numerals refer to like elements in the 1 and 10 series of reference numerals, unless otherwise noted. The hemispherical shape of sheath 410 has a superior opening 427 through a polar region and defined by inner rim 432 and an inferior opening opposite the polar region as defined by outer rim 411. Superior opening 427 has a smaller diameter than that of inferior opening 432. Sheath 410 also includes a convex surface 426 designed to face a shell when assembled as part of an implant assembly, the convex surface extending between inner rim 432 and outer rim 412. Opposite convex surface 426 is a concave surface 429 that defines a cavity and is designed to face a liner when assembled as part of an implant assembly. Sheath 410 includes a plurality of holes 430 that each extend through the sheath from convex surface 426 to the concave surface 429. Included among the plurality of holes is a first circumferential row of holes 434, a second circumferential row of holes 436, and a third circumferential row of holes 438, each being offset to a different extent from the annular rim 411. The holes in each row are spaced at equal intervals. And, spacing of the holes in row 434 is different from the spacing of the holes in row 436, which in turn is different from the spacing of the holes in row 438. Additionally, opening sizes of the holes on convex surface 426 or concave surface 429 varies among holes 434, holes 436 and holes 438, as shown in FIG. 15, for example. Each hole of the plurality of holes 430 is aligned along a central longitudinal axis (e.g., 431) that is perpendicular to and passes through a central longitudinal axis 425 of sheath 410, where the axis 425 passes through centers of the superior and inferior openings. Additionally, each hole of the plurality of holes 430 may be coaxial with the longitudinal axis of an opposite hole of the plurality of holes 430 by being located directly across therefrom. In other words, a central axis through each hole of the plurality of holes 430 may be concentric with a central axis through one other hole, as shown in FIG. 15, with a specific example indicated by axis 431. This arrangement of the holes through the sheath may simplify fabrication of sheath 410 as two holes can be formed along one drill axis. In variations of sheath 410, each row of holes may have holes spaced at different or irregular intervals. Similarly, variations may include hole openings of different sizes than that shown in FIGS. 14-17 and hole angulation that varies from the depicted embodiment.

In yet another embodiment, a modified liner assembly 401A includes a liner 402 and sheath 410 attached thereon, as shown in FIG. 17. The liner may have a similar structural design as the liner described herein. Sheath 410 is designed to receive and affix to the liner in a similar manner as the other sheaths and liners described herein such that sheath 410 connects with exterior features of the liner. In this manner, sheath 410 is combined with a liner to create a modified liner configured to be implanted into a shell similar to the other modified liners described herein.

In another embodiment, a prosthetic implant assembly (not shown) includes a shell, a liner, and a sheath 410 that is disposed between the shell and the liner. In other words, a modified liner comprising sheath 410 may be disposed within a shell to create a prosthetic implant assembly, similarly to the other prosthetic implant assemblies described herein. For example, a prosthetic implant assembly may include a shell similar to shell 303 and modified liner assembly that includes a liner like liner 302 and sheath 410 disposed thereon. As with other implant assembly embodiments, the modified liner may be fixed in place relative to the shell. Such fixation can be tilted and slanted at any angle ranging from 0 degrees up to 90 degrees as necessary. In this manner, the modified liners may be better suited for the biomechanics of a patient. Additionally, as with the other embodiments, while sheath 410 is shown to be compatible with a specific shell and liner in the figures, it should be understood that the sheath may have a modular design that will allow it to be used with a large variety of shells and liners of different shapes and sizes.

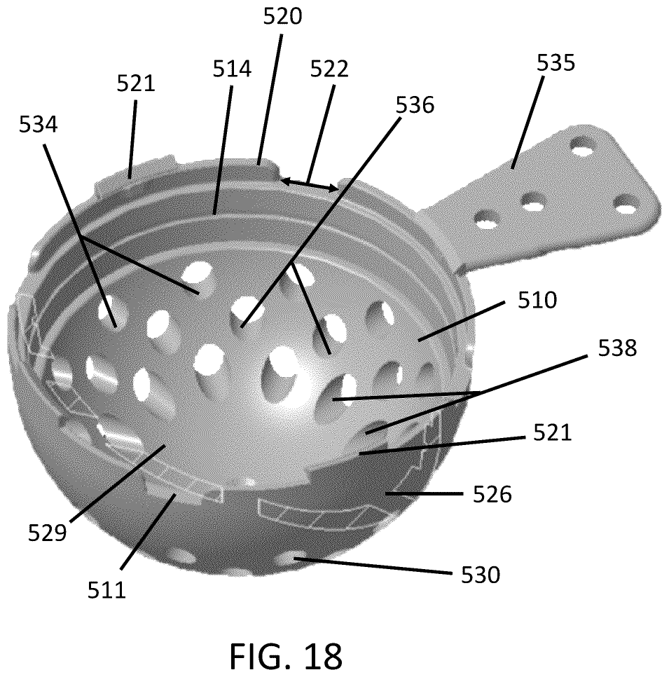

In another embodiment, sheath 510 is shown in FIG. 18. In FIG. 18, reference numerals of the 500 series of numerals refer to like elements in the 400 series of numerals in FIGS. 14-17, unless otherwise noted. Sheath 510 may include many of the same features as sheath 410. Sheath 510 includes a plurality of holes 530 and has a closed polar region such that sheath 510 appears similar to a colander-like bowl structure. The longitudinal axis of each hole of the plurality of holes 530 is perpendicular to and passes through a central longitudinal axis of the sheath and may be coaxial with the longitudinal axis of another hole of the sheath directly across from the hole. This hole alignment feature allows for more efficient fabrication of sheath 510 as two holes can be drilled along one axis. Similar to the embodiment shown in FIG. 14-17, the plurality of holes 530 are disposed in a first circumferential row of holes 534, a second circumferential row of holes 536, and a third circumferential row of holes 538 with each of the plurality of holes 530 extending through a thickness of the sheath between convex surface 526 and concave surface 529.

Sheath 510 also includes various attachment features such as lips 521 and a flange 535. Lips 521 extend from tabs 520, which in turn extend from annular rim 511. Flange 535 extends radially outward from annular rim 511 of the sheath in a direction away from the body of the sheath. The flange may have a generally flat shape with opposing planar surfaces and may optionally pass through a plane that passes through annular rim 511. Flange 535 may also include one or more holes therethrough, as shown in FIG. 18, for example. Such holes may function as holes for receiving fasteners to secure the flange to bone. Flange 535 may also have dimensions customized to accommodate available bone in a patient or be made from materials with a modulus of elasticity that allows for deformation of the flange. In some examples, flange 535 may be made from titanium such as Ti64. The use of such material for the flange provides a flange rigid enough to maintain fixation with bone, yet ductile enough to bend as necessary to accommodate the contours of the patient anatomy. In this manner, flange 535 provides an additional manner of anchoring the sheath and any accompanying implant components into bone. Such additional anchorage may be particularly advantageous in complex revision surgeries. It is further contemplated that variants of the other sheaths described herein may also include a flange similar to flange 535.

In yet another embodiment, a modified liner assembly includes a liner and sheath 510 attached thereon. The liner (not shown) may have a similar structural design as other liners described herein. Sheath 510 is designed to be received on and affix to the liner in a similar manner as the other sheaths and liners described herein. Engagement features near and extending from annular rim 511 of sheath 510 may engage with complementary features on the liner to provide a fixed connection. Additionally, lips 521 of sheath 510 may be disposed over a rim of the liner to secure the liner therein. In this manner, sheath 510 can be combined with a liner to create a modified liner configured to be implanted into a shell.

In another embodiment, a prosthetic implant assembly includes a shell and a modified liner. The modified liner includes a liner and sheath 510 that is received on and engaged to the liner. The modified liner comprising sheath 510 may be disposed within and secured to a shell with adhesive, such as bone cement, to create a prosthetic implant assembly, similar to the other prosthetic implant assemblies described herein.

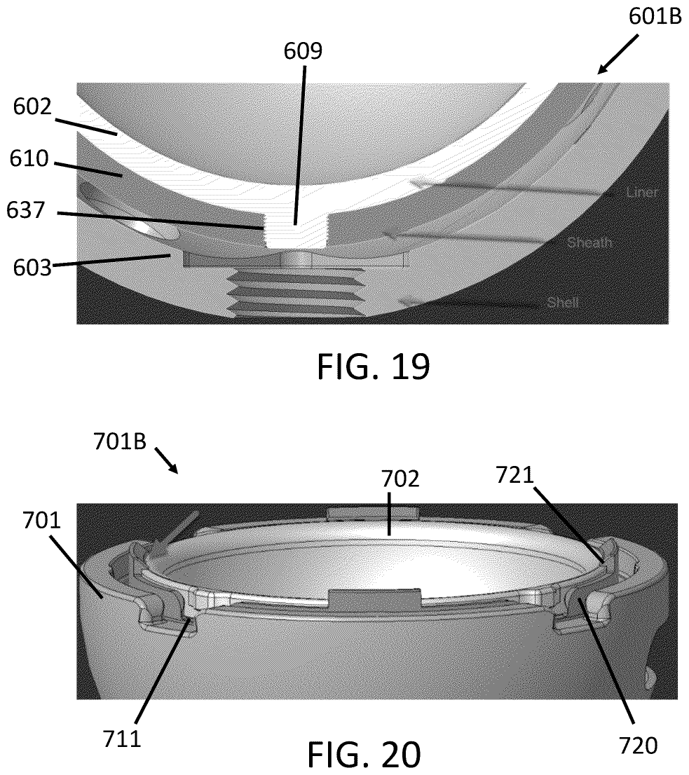

With reference to embodiments including at least the sheath and the liner, it should be appreciated that there are a multitude of ways to ensure attachment of the sheath to the liner. The following non-limiting examples of attachment mechanisms can be used separately or in combination with any sheath contemplated by the present disclosure. In one example, as mentioned above with respect to sheath 110, a notch (e.g., notch 123 of sheath 110) on an inside surface of an annular band at the rim of the sheath may engage with a projection 104 on an outer surface of the liner proximate the rim. The liner may be impacted over the sheath in such position to interdigitate the components. In another example, the modular sheath may include a threaded hole through a polar region of the sheath, such as hole 637 of sheath 610 shown in FIG. 19. Hole 637 may be sized and otherwise configured to receive a post of a liner, such as threaded post 609, extending from a liner such that the liner and the sheath are threadably attached. Reference numerals of the 600 series of numerals refer to like elements in the 1 and 10 series of numerals, unless otherwise noted. In yet another example, the modular sheath may include a plurality of lips (e.g., 521, 721) spaced apart along a rim (e.g., 511, 711) of the sheath and facing away from a body of the sheath, such as is shown in FIG. 20. Such lips may extend from tabs (e.g., tabs 120, 320, 420, 820) of the sheath. The lips 521, 521 include inward facing protrusions shaped so that when positioned over a liner 702, such lips hook onto the rim of the liner, as shown in FIG. 20. Reference numerals of the 700 series of numerals refer to like elements in the 500 series of numerals, unless otherwise noted. In another example, as mentioned above and shown in FIG. 18, the modular sheath may include a flange that extends away from the annular rim of the modular sheath. Such flange may have one or more holes therethrough for the receipt of a fastener such as a screw that may be used to anchor the flange to bone. The implant assemblies 601B, 701B respectively shown in FIGS. 19 and 20 each include specific components for securement of a sheath to a liner. The features of implant assembly 601B may be incorporated into any one of the embodiments contemplated by the present disclosure and similarly, the features of implant assembly 701B may be incorporated into any one embodiment of the present disclosure. In some examples, an implant assembly may include the features of both assemblies 601B and 701B. While the embodiments of FIGS. 19-20 are described as being different embodiments, it should be understood that the features and components shown in FIGS. 19-20 can also be part of one embodiment. Additionally, engagement features or mechanisms included in implant components of the Trident® II Tritanium Acetabular System by Stryker® may be incorporated into any one of the embodiments contemplated by the present disclosure.

As described herein and shown in the figures, some embodiments of the modular sheath include a network of interconnected segments, e.g., ribs designed to be entombed in an adhesive, e.g., but not limited to, bone cement, after being attached to a liner and placed in a shell. In this manner, the sheath securely anchors the liner to the shell by forming a strong connection between the adhesive and the liner.

The sheaths described herein may have a variety of thicknesses, but may be customized so that a variety of liner sizes may be accommodated for a particular joint. In some examples, the sheath may be 1.5 mm thick to minimize a volume occupied by the sheath and thus provide a maximal amount of space for a liner within a shell. In other examples, the sheath may have a thickness greater than 1.5 mm. For example, the sheath may be 1.6, 1.7, 1.8, 1.9, 2.0 mm thick or greater. In other examples, the sheath may have a thickness of 2.25 mm, 2.5 mm, or other values. One factor respecting an upper limit on a thickness of the sheath involves consideration of whether it is expected that the sheath, when used to form a modified liner and combined with a shell, is expected to have adequate post-operative function given the thickness of cement mantle that will be applied and cured for such an assembly. With thicknesses as described above, the sheath advantageously maintains adequate strength to anchor a liner within the shell.

As to materials, the sheath, liner and shell as contemplated by the present disclosure may be made of a variety of materials. The sheaths described herein may be made from various metals and/or polymeric materials that are safe for implantation into the human body. Metals may include, but are not limited to, titanium, titanium alloys, stainless steel, or stainless-steel alloys. Polymeric materials may include various plastics that are biocompatible for implantation in a patient. As for the modified liner and prosthetic implant assemblies described herein, the shells and liners for such assemblies may be made from various metals, polymeric materials, and/or ceramics.

In another aspect, the present disclosure relates to kits including one or more of implant components and surgical tools for implanting such implant components. The implant components may be part of a prosthetic ball-and-socket implant assembly, such as an assembly for hip or shoulder replacement. The kit may include one or more sheaths, such as one of the sheaths described above, along with multiple liners having different sizes and/or shapes. Alternatively, a kit may include a shell, a sheath, and at least one liner. Or a kit may include one of the sheaths described herein and two or more liners that differ in some aspect such as size and/or shape. A kit may also include an adhesive suitable to adhere the liner and sheath to the shell such as bone cement. In some examples, the kit may further include tools for mixing and applying an adhesive.

In another aspect, the present disclosure relates to a method of manufacturing a sheath or multiple components of an implant assembly. In some examples, the implant components described in the present disclosure may be manufactured using traditional manufacturing techniques, such as injection molding and other subtractive manufacturing techniques known to persons of skill in the art. In some examples, the sheaths contemplated by the present disclosure may be fabricated using additive manufacturing (i.e., 3D printing), techniques. Such techniques may include, but are not limited to, binder jetting, selective laser melting (SLM), selective laser sinter (SLS), electron beam melting (EBM), direct metal laser sintering (DMLS), etc., and by other fabrication methods that utilize computer aided manufacturing (CAM) techniques. Furthermore, the sheaths may be formed using one or more of the above techniques and/or the additive manufacturing fabrication techniques as described in U.S. Pat. Nos. 4,863,538, 5,017,753, 5,076,869, 4,944,817, 10,614,176, 11,534,307, 11,737,880, and 7,537,664, the entire disclosures of which are incorporated by reference. Additive manufacturing of the sheath is advantageous as it may be used to produce sheaths with a desired overall thickness and rib sizes with high accuracy and within desired tolerances. It should be appreciated that any one of the embodiments of the sheath contemplated by the present disclosure may be fabricated through such additive manufacturing techniques. Further, the liners and shells contemplated by the present disclosure may also be fabricated through the aforementioned additive manufacturing techniques. Additionally, in other examples, the sheath, liner and/or shell may be fabricated using a combination of additive and traditional manufacturing techniques.

In yet another aspect, the present disclosure relates to a method of assembling components of a prosthetic implant that includes a sheath. In one embodiment, the method may include inserting and attaching a liner to a sheath, e.g., sheath 10, 110, 210, 310, 410, 510, to form a modified liner. For example, when the liner includes a circumferential ring, the liner is inserted into the sheath until the circumferential ring of the liner engages a mating groove on the band portion of the sheath (e.g., groove 14, 114, 214, 314, 414, 514). Such engagement may require physical impaction of the liner into the sheath. When the sheath includes tabs (e.g., tabs 120, 420, 520) extending from its annular rim, then liner-removal components (e.g., projections 4, 104, 204, 304) extending from the liner are aligned with cutouts (e.g., recesses 122, 422, 522) or notches (e.g., notches 123, 423) before or during the insertion of the liner into the sheath.

In other embodiments, the preceding method may also include taking the modified liner assembled according to the preceding steps and securing the modified liner to an acetabular shell. To secure the modified liner, an adhesive is applied onto a surface of a cavity of the shell, the modified liner, or both. Next, the modified liner is inserted into the shell until interconnected segments of the sheath are encased in the adhesive, and simultaneously or shortly after this step, the modified liner is oriented to a desired angle with respect to the shell. In some variations of these embodiments, the method may continue with one or more steps of the assembly of the femoral implant, including the disposal of a prosthetic femoral head of the femoral implant into a cavity of the liner. The step of placing the prosthetic femoral head into the liner may be performed at any stage of the assembly process.

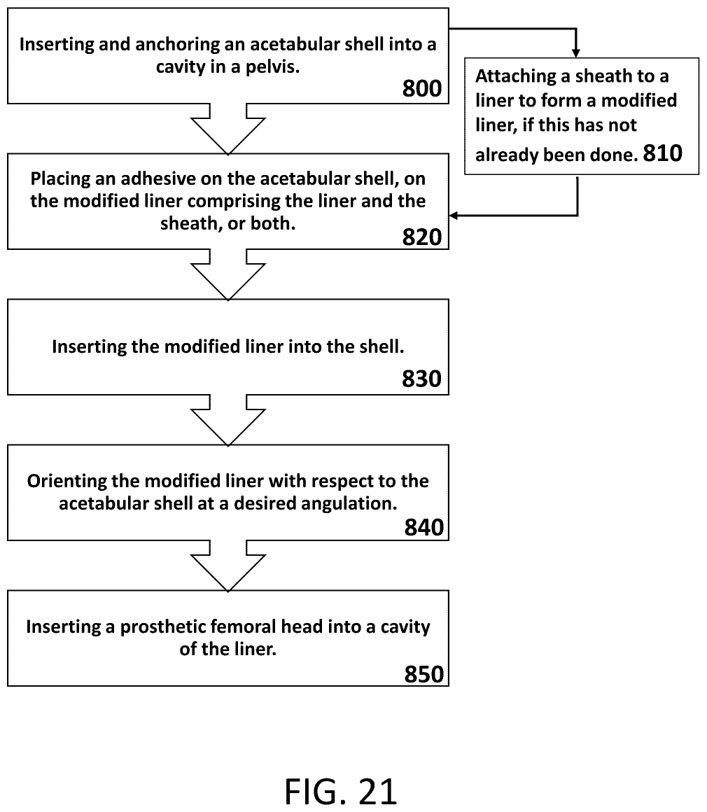

In another aspect, the present disclosure relates to a method of implanting a prosthetic joint assembly into a ball and socket joint of a patient. In one embodiment, the method relates to implantation of a hip implant during a primary surgery, as shown in FIG. 21. The method may include a step 800 of inserting and anchoring an acetabular shell into a cavity in a pelvis. In some variations of this method, the method may also include a step 810 of attaching a sheath to a liner to form a modified liner, if this has not already been done. Once the shell is inserted and secured, a step 820 is performed where an adhesive is placed on the acetabular shell, on a modified liner including a liner and sheath, such as sheath 10, 110, 210, 310, or 410, or both the shell and the modified liner. Next, step 830 is performed where the modified liner is inserted into the shell. Once the modified liner has been inserted, step 840 includes orientation of the modified liner with respect to the shell at a desired angulation. With modified liner secured in place within the shell and the adhesive cured so that the modified liner is immovable relative to the shell, a step 850 may be performed including insertion of a prosthetic femoral head into a cavity of the liner.

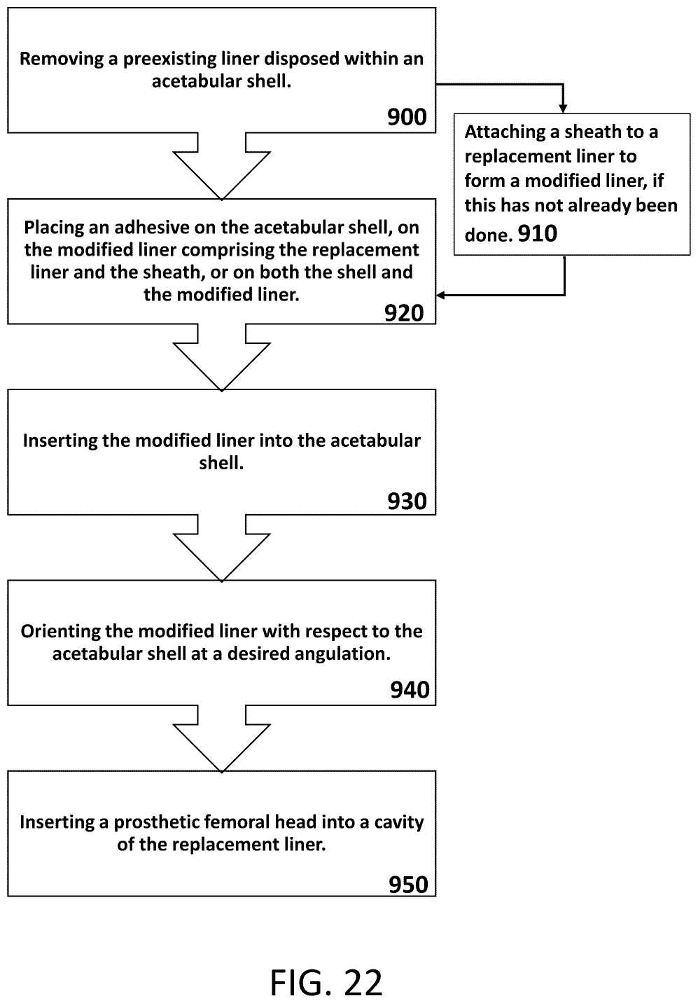

In yet another aspect, the present disclosure relates to a method of replacing an existing liner in an existing hip implant within a patient, as shown in FIG. 22. This procedure may also be referred to as a revision surgery. The method may commence with a step 900 of removing a preexisting liner disposed within an acetabular shell, where the shell is preexisting within a patient. When the existing liner includes one or more removal features such as protrusions extending radially outward from a rim, a prying tool can be inserted under such protrusions to pry out the replacement liner from the implanted shell. With the original liner removed, the acetabular shell is prepared to receive a replacement liner in the form of a new liner (e.g., liner 10, 110, 210, 310, or 410). In some variations, this method may include an optional step 910 of attaching the sheath to the replacement liner to form a modified liner, if the modified liner has not already been assembled. With the acetabular shell ready to receive the replacement liner, a step 920 is performed where an adhesive is placed on the acetabular shell, on the modified liner including the replacement liner and sheath (e.g., sheath 10, 110, 210, 310, or 410), or both the shell and the modified liner. Next, step 930 includes inserting the modified liner into the acetabular shell. Once the modified liner is inserted into the acetabular shell, step 940 includes orienting of the modified liner with respect to the shell at a desired angulation. With modified liner secured in place within the shell and the adhesive cured so that the modified liner is set in place therein and immovable relative to the shell, a step 950 may be performed including insertion of a prosthetic femoral head into a cavity of the replacement liner.

The methods of assembly described above may also optionally include an additional step of attaching the sheath to the liner. For example, a liner having a threaded post (e.g., post 609) is threaded into a sheath having a threaded hole (e.g., hole 637) that is configured to receive the threaded post as shown in FIG. 19. Or alternatively, after a liner has been partially inserted into a sheath, the sheath is impacted onto the liner until lips (e.g., lips 621) of the sheath extend over a rim of the liner as shown in FIG. 20. Another example includes a liner being inserted into the sheath such that the sheath engages with locking features protruding from the exterior of the liner. The securing of the shell may include bone screws being inserted into screw holes on the shell and the bone. The modified liner may be formed by impacting the sheath onto the liner such that the sheath engages the taper near the rim of the liner. The orienting of the modified liner may include the modified liner being arranged such that the rim of the shell and the rim of the liner are not parallel, as shown in FIG. 4 and FIG. 10. The angulation of the liner relative to the shell may be any angle as desired by the surgeon or other medical professional. For example, 5 degrees, 10 degrees, and so on, up to even 80 or 90 degrees in extreme cases.

It is to be understood that the disclosure set forth herein includes any possible combinations of the particular features set forth above, whether specifically disclosed herein or not. For example, where a particular feature is disclosed in the context of a particular aspect, arrangement, configuration, or arrangement, that feature may also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A prosthetic implant assembly, comprising:
a modified liner configured for use in a hip joint, the modified liner comprising:
a sheath having an annular rim, a plurality of longitudinal ribs extending from the annular rim, and a first cross-rib spaced apart from the annular rim and intersecting the plurality of longitudinal ribs, the first cross-rib defining a closed loop,
wherein each longitudinal rib of the plurality of longitudinal ribs is curved such that the plurality of longitudinal ribs collectively define a partially spherical surface, and
wherein the annular rim, the plurality of longitudinal ribs and the first cross-rib define a plurality of openings between the annular rim and the first cross rib; and
a liner configured to be received in the sheath.

2. The prosthetic implant assembly of claim 1, wherein the sheath includes a second cross-rib defining a closed loop and spaced apart from the first cross-rib, the second cross-rib intersecting the plurality of longitudinal ribs.

3. The prosthetic implant assembly of claim 2, wherein the plurality of longitudinal ribs, the first cross-rib and the second cross-rib define a webbed structure such that a majority of the plurality of openings are quadrilateral.

4. The prosthetic implant assembly of claim 1, wherein the sheath further comprises a plurality of cross-ribs in addition to the first cross-rib, each cross-rib of the first cross-rib and the plurality of cross-ribs being evenly spaced with respect to an adjacent cross-rib.

5. The prosthetic implant assembly of claim 4, wherein each end of each longitudinal rib of the plurality of longitudinal ribs is attached to the annular rim of the sheath, and the plurality of cross-ribs extend circumferentially around the sheath.

6. The prosthetic implant assembly of claim 1, wherein the cross-rib varies in distance from the annular rim along its length.

7. The prosthetic implant assembly of claim 1, wherein each opening of the plurality of openings is a cuboid shape.

8. The prosthetic implant assembly of claim 1, wherein the annular rim has a band extending therefrom.

9. The prosthetic implant assembly of claim 8, wherein, in a direction of a length of the longitudinal rib, a dimension of the band is greater than each of the cross-ribs.

10. The prosthetic implant assembly of claim 1, wherein the sheath has a hemispherical shape.

11. The prosthetic implant assembly of claim 10, wherein the annular rim is located at an equatorial region of the hemispherical shape.

12. The prosthetic implant assembly of claim 1, wherein the sheath is a truncated hemisphere having a first end opening and a second end opening, the first end opening defining an inner diameter of the annular rim, the inner diameter of the annular rim being larger than that of the second end opening.

13. The prosthetic implant assembly of claim 12, wherein the second end opening defines a jagged edge configured to flex around the liner inserted therein.

14. The prosthetic implant assembly of claim 12, wherein the first and second end openings are concentric.

15. The prosthetic implant assembly of claim 1, wherein the liner includes a lip that extends over the annular rim of the sheath.

16. A prosthetic implant assembly comprising:
a sheath having an annular rim with a first diameter, a plurality of longitudinal ribs each extending along a curve from the annular rim, and a cross-rib with a second diameter intersecting the plurality of longitudinal ribs,
wherein the second diameter is smaller than the first diameter,
wherein an implant-receiving side of each longitudinal rib of the plurality of longitudinal ribs defines a portion of a cavity, each longitudinal rib of the plurality of longitudinal ribs having a convex side opposite the implant-receiving side, and
wherein the plurality of longitudinal ribs and the cross-rib define a plurality of intersection points between the plurality of longitudinal ribs and the cross rib; and
a liner configured to be received in the cavity of the sheath.

17. The prosthetic implant assembly of claim 16, further comprising an implanted acetabular shell configured to receive a modified liner comprising the sheath and the liner.

18. The prosthetic implant assembly of claim 16, wherein the sheath includes a plurality of cross-ribs.

19. The prosthetic implant assembly of claim 16, wherein the cross-rib includes a plurality of U-shaped segments.

20. A kit for acetabular implant revision, comprising:
a plurality of liners, at least two liners of the plurality of liners being different from each other; and
a sheath adapted to receive each liner of the plurality of liners, the sheath having an at least partial cup shape with a cavity-facing surface and a convex surface opposite the cavity-facing surface, a plurality of first openings extending circumferentially parallel to an annular rim of the sheath and a plurality of second openings extending circumferentially parallel to the annular rim, wherein the plurality of first openings are at a different distance from the annular rim compared to the plurality of second openings and the plurality of first openings are different from the plurality of second openings.
